(19) United States
(12) Patent Application Publication
(10) Pub. No.: US 2011/0125260 A1
(43) Pub. Date: May 26, 2011

(54) ARTIFICIAL CORNEA

(75) Inventor: Tueng T. Shen, Redmond, WA (US)

(73) Assignee: UNIVERSITY OF WASHINGTON, Seattle, WA (US)

(21) Appl. No.: 12/912,529

(22) Filed: Oct. 26, 2010

Related U.S. Application Data
(63) Continuation of application No. PCT/US2009/042418, filed on Apr. 30, 2009.
(60) Provisional application No. 61/049,254, filed on Apr. 30, 2008.

(51) Int. Cl. A61F 2/14; B29D 11/00

(52) U.S. Cl. 623/5.16; 264/2.5

(57) ABSTRACT

The present invention provides an artificial cornea (keratoprosthesis) that may be implanted into a patient in need thereof. The artificial cornea comprises a rigid transparent central core surrounded by a peripheral skirt comprising a porous hydrogel. Methods of making such artificial corneas are also provided.

### CROSS-REFERENCES TO RELATED APPLICATIONS

This application is a continuation of International Patent Application No. PCT/US2009/042418, filed Apr. 30, 2009, which claims the benefit of U.S. Provisional Application No. 61/049,254, filed Apr. 30, 2008, both of which applications are expressly incorporated herein by reference in their entirety.

### BACKGROUND OF THE INVENTION

The cornea, a transparent tissue covering the front of the globe, is the window of the eye. It is responsible for approximately 70% of the eye's refractive power, and it serves as the eye's protective barrier. Corneal blindness, which involves the opacification of the transparent corneal tissue, affects 12 million people today, and it is the second most treatable blindness in the world. The global economic loss of productivity from blindness is estimated at $19 billion in 2000 and is expected to reach $50 billion in 2020 if no effective intervention is implemented.

The standard treatment for corneal blindness is corneal transplant surgery, where fresh human donor cornea is sutured to the host to replace the damaged host cornea. Despite the fact that corneal blindness is relatively treatable, only about 100,000 corneal transplant surgeries are performed each year, leaving most corneal blind patients untreated. This disparity results from the limited availability of human donor tissue in most parts of the world. Additionally, most cases of severe corneal blindness (mostly found in developing nations) are not amenable to conventional corneal transplant surgery using human tissue.

Corneal transplant surgery using high quality human donor corneal grafts has been performed successfully since 1905. This approach can often restore good vision and often requires only local immune suppression. In the United States, approximately 39,000 corneal transplant surgeries are performed each year. Post-operative visual outcomes depend, in part, on a surgeon's skill and the quality of donor tissues. The demand for high quality donor corneas is still increasing as a result of longer life expectancies; however, the supply is decreasing. For example, the availability of acceptable donor tissue is expected to decrease significantly with the increased popularity of LASIK refractive surgery (over one million per year), because these surgically treated corneas are unacceptable as donor tissues.

Survival of a corneal graft is also dependent upon the cause of host blindness. Patients with non-complicated corneal diseases such as inactive central cornea scars and keratoconus usually have good prognoses (e.g., 80% graft survival at two years; 65% at five years). However, others with severe traumatic injuries, immunologic disorders (such as Stevens-Johnson Syndrome), and corneal limbal stem cell deficiency have very limited success with conventional cornea transplants.

All transplanted corneal grafts are at risk for rejection, irregular astigmatism (from suture use), and infection. Graft rejection is one major concern after corneal transplant surgery, and patients may require long-term local immune-suppression. Once rejection occurs, repeat transplant surgery has increased risks for further rejections and graft failure.

Currently available artificial corneas for human use include the Boston Keratoprosthesis (Boston KPro) and AlphaCor™. For several reasons, both corneal substitutes have limited clinical use. For example, poor integration of such artificial corneas with host tissue can lead to a high risk of catastrophic infection and extrusion. Additionally, Boston KPro depends upon human donor tissue, which is a limitation as noted above. Further limitations of AlphaCor™ lie in its two-stage surgical implementation and its relatively slow visual recovery time.

The disadvantages associated with presently available artificial corneas demonstrates the need for improved devices. A high-performance, well-integrated synthetic cornea could dramatically improve the quality of treatment of corneal blindness while simultaneously bringing cost-effective and safe treatment within the reach of a much larger fraction of corneal blindness sufferers.

### SUMMARY OF THE INVENTION

The present invention provides a keratoprosthesis (artificial cornea) that may be implanted into a patient. More particularly, the present invention provides, in certain embodiments, a keratoprosthesis, comprising: (a) a rigid transparent central core; and (b) a peripheral skirt comprising a porous hydrogel.

Methods of making aspects of a keratoprosthesis are also contemplated, such as a method of making a peripheral pre-form skirt of keratoprosthesis comprising a porous hydrogel, the method comprising: (a) heat sintering monodisperse poly(methyl methacrylate) porogens to create a template; (b) combining the template with a solution comprising a monomer or mixture of monomers; (c) polymerizing the solution; and (d) removing the template to reveal a porous hydrogel.

In certain embodiments, the present invention provides a method of making a keratoprosthesis comprising: (a) forming or placing a porous hydrogel in the periphery of an injection molding apparatus to form a peripheral pre-form skirt; (b) injecting a solution comprising a monomer or a mixture of monomers into the injection molding apparatus such that the solution occupies the area inside the skirt; and (c) polymerizing the monomer(s) to generate a transparent central core.

In other embodiments, method of treatment are provided, such as a method of treating corneal blindness comprising implanting a keratoprosthesis of the present invention into a patient.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A and 1B are views of a keratoprosthesis of the present invention.

FIG. 1A is a top view. FIG. 1B is a cross-sectional view.

FIG. 2 is a side view of a molding apparatus, which may be used in the manufacture of a keratoprosthesis of the present invention.

FIG. 3 is a three-dimensional view of a molding apparatus which may be used in the manufacture of a keratoprosthesis of the present invention.

FIGS. 4A, 4B, and 4C are side views of a molding apparatus, which may be used in the manufacture of a keratoprosthesis of the present invention. FIG. 4A shows placement of a peripheral skirt comprising a porous hydrogel. FIG. 4B shows the peripheral skirt in combination with monomers that occupy the central core. FIG. 4C shows polymerization of the monomers that results in a finished keratoprosthesis.

FIG. 5 is a photograph of a porous hydrogel produced using the porogen template technique discussed herein.

### DETAILED DESCRIPTION OF THE INVENTION

The present invention provides a one-piece artificial cornea as well as methods of making and using such corneas. Artificial corneas of the present invention offer, for example, reduced surgery implementation times, superior visual outcomes, and shorter recovery periods compared to artificial corneas presently employed. In addition, artificial corneas of the present invention do not rely on donor corneal graft tissue in their construction or implantation. As used herein, an “artificial cornea” is equivalent to a “keratoprosthesis.”

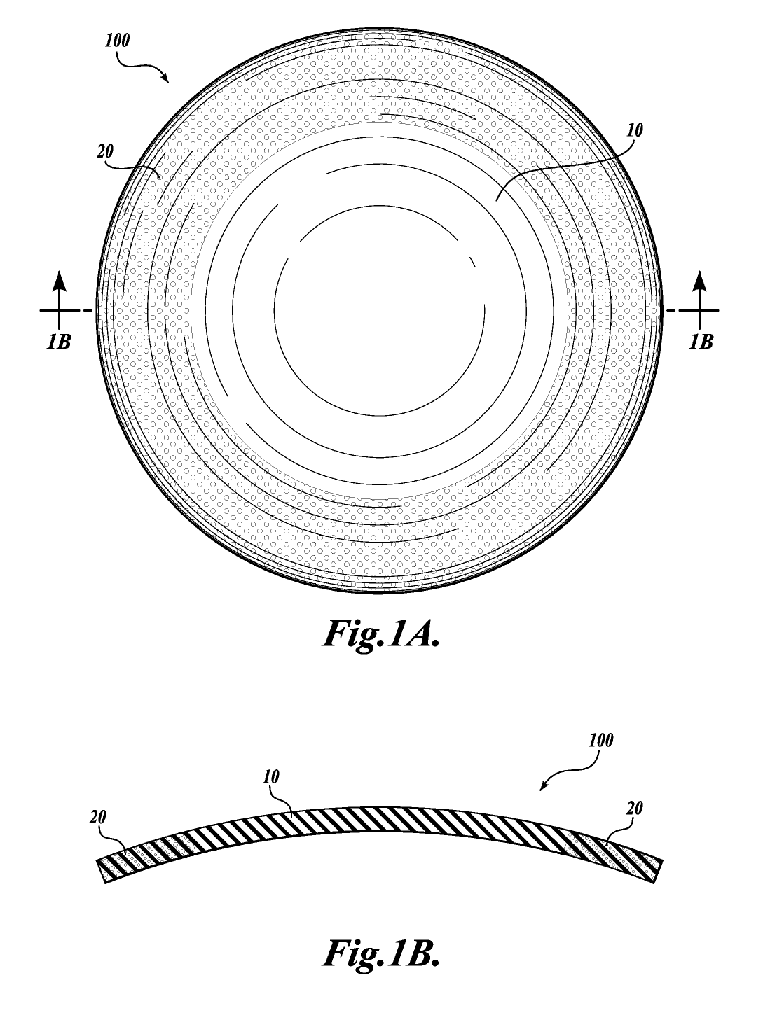

Accordingly, the present invention contemplates a keratoprosthesis, comprising: (a) a rigid transparent central core; and (b) a peripheral skirt comprising a porous hydrogel. In certain embodiments, a keratoprosthesis may be described as having a radially extended transparent central core that is defined by an annular peripheral skirt comprising a porous hydrogel (see, e.g., FIG. 1A).

In certain embodiments, the term “rigid” refers to a transparent central core having a Young's modulus value that ranges between 500-3500 MPa. In certain embodiments, the transparent central core has a Young's modulus of about, at most about, or at least about 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, or 3500 MPa, or any range derivable therein. For example, the range may be 1800-3100 MPa. Methods of measuring Young's modulus are well-known in the art.

The term “rigid” may also refer to the Shore D hardness of a transparent central core. In certain embodiments, the transparent central core has a Shore D hardness that ranges between 60-100. In certain embodiments, the Shore D hardness is about, at most about, or at least about 60, 65, 70, 75, 80, 85, 90, 95, or 100, or any range derivable therein. For example, the range may be 80-90. Methods of measuring Shore D hardness are well-known in the art.

The term “rigid” may also refer to a tensile strength of a central core. In certain embodiments, the tensile strength ranges from 30-70 MPa. In certain embodiments, the transparent central core has a tensile strength that is about, at least about, or at most about 30, 35, 40, 45, 50, 55, 60, 65, or 70 MPa, or any range derivable therein. Methods of measuring tensile strength are well-known in the art.

In any embodiment, any combination of Young's modulus, Shore D hardness, and/or tensile strength may be used to describe a rigid transparent central core. For example, a rigid transparent central core may have a Young's modulus of 1800-3100 MPa, a Shore D hardness of 80-90, and a tensile strength of 30-70 MPa.

As used herein, “transparent” refers to the ability to transmit a substantial portion of visible light through the central core, such as greater than or equal to 80% of incident light. In certain embodiments, about or at least about 85%, 90%, 95%, or 99%, or more of incident light is transmitted.

Any material that satisfies any one or more of the rigid parameters described above may be employed in a transparent central core of the present invention. In certain embodiments, the transparent central core comprises poly(methyl methacrylate) (PMMA). In more specific embodiments, the transparent central core consists essentially of PMMA.

In certain embodiments, the transparent central core is not a hydrogel composed essentially of a biocompatible hydrophilic polymer. As used herein, the term “hydrogel” refers to a three-dimensional polymer that measurably swells in an aqueous solution due to the absorbance of water. A hydrogel includes water or an aqueous solution as part of its structure. In certain embodiments, a polymer that swells at least 10% in an aqueous solution when it is fully hydrated due to the absorbance of water is referred to as a hydrogel.

In certain embodiments, the central core is not a hydrogel composed essentially of a biocompatible hydrophilic polymer as described in U.S. Pat. No. 5,300,116, which is incorporated herein by reference in its entirety. For example, a central core may not be composed essentially of 2-hydroxyethyl methacrylate alone or in combination with a hydrophobic polymer (e.g., a hydrophobic aliphatic methacrylate or hydrophobic aliphatic acrylate).

The diameter of the transparent core may be greater than 3 mm. In certain embodiments, the diameter ranges from about 5-8 mm. For example, the diameter may be about, at most about, or at least about 5.0, 5.5, 6.0, 6.5, 7.0, 7.5, or 8.0 mm, or any range derivable therein. In particular embodiments, the diameter of the central core is 6.5 mm.

Regarding the thickness of keratoprostheses of the present invention, the thickness of the transparent central core and the peripheral skirt may be identical. In certain embodiments, the thickness of the core is ±0-20% of the thickness of the peripheral skirt. For example, the relative thickness of the core may be about, at most about, or at least about 0%, 1%, 2%, 3%, 4%, 5%, 6%, 7%, 8%, 9%, 10%, 11%, 12%, 13%, 14%, 15%, 16%, 17%, 18%, 19%, or 20% of the thickness of the peripheral skirt, or any range derivable therein. In certain embodiments, the thickness of the keratoprosthesis is uniform: that is, the difference in thickness between the core and the skirt is 0%. In certain embodiments, the core thickness, the skirt thickness, or the uniform thickness of the keratoprosthesis is about, at least about, or at most about 50, 55, 60, 65, 70, 76, 80, 85, 90, 95, 100, 105, 110, 115, 120, 125, 130, 135, 140, 145, 150, 155, 160, 165, 170, 175, 180, 185, 190, or 200 μm, or any range derivable therein.

The thickness of the core, skirt, or keratoprosthesis as a whole may be greater at one part and less at another. For example, a core may be thicker in the center of the core and then taper towards the peripheral skirt. Typically, the thickness of a keratoprosthesis is about the same as that of the host cornea tissue.

The transparent central core may exhibit a range of diopter values. For example, the diopter value may range between about 10-70 diopters. In certain embodiments, the diopter value is about, at most about, or at least about 10, 15, 20, 25, 30, 35, 40, 45, 50, 55, 60, 65, or 70 diopters. Methods of measuring diopter values are well-known in the art.

The transparent central core may further comprise, for example, a bacterial-resistant biofilm. As used herein, a “bacterial-resistant biofilm” refers to a biofilm that exhibits an adverse effect on bacteria, particularly disease-causing bacteria. The bacteria may be Gram positive or Gram negative.

As noted above, a keratoprosthesis of the present invention comprises a peripheral skirt comprising a porous hydrogel. The skirt provides a platform for integration into the host through suturing.

As used herein, “porous” refers to a peripheral skirt comprising an array of pores. Typically, substantially all the pores in the biomaterial have a similar diameter. As used herein, the term “substantially all the pores” refers to at least 90% of the pores in the biomaterial, such as at least 95% or at least 97% of the pores. The term “diameter of a pore” refers to the longest line segment that can be drawn that connects two points within the pore, regardless of whether the line passes outside the boundary of the pore.

Two pores have a “similar diameter” when the difference in the diameters of the two pores is less than about 20% of the larger diameter. Typically, the mean diameter of the pores is between about 10 and about 100 nm, such as between about 25 and 75 nm or between about 30 and 60 nm. In some embodiments, the mean diameter of the pores is between about 30 and 40 nm. In certain embodiments, the mean diameter of the pores is about, at most about, or at least about 10, 15, 20, 25, 30, 35, 40, 45, 50, 55, 60, 65, 70, 75, 80, 85, 90, 95, or 100 nm, or any range derivable therein.

The pores may have any suitable shape. For example, the pores may be roughly, or perfectly, spherical. Other suitable pore shapes include, but are not limited to, dodecahedrons (such as pentagonal dodecahedrons) and ellipsoids. In some embodiments, substantially all the pores have a roundness of at least about 0.1, such as at least about 0.3 or at least about 0.7. As used herein, “roundness” is defined as (6×V)/(pi×D³), where V is the volume and D is the diameter. For example, a sphere has a roundness equal to 1.

In the array of pores, substantially all the pores are each connected to at least four other pores. In some embodiments, substantially all the pores are connected to between about 4 to 12 other pores, such as between about 4 to 7 other pores. Substantially all the connections between the pores have a diameter that is between about 15% and about 40%, such as between about 25% and about 30%, of the mean diameter of the pores. As used herein, the term “diameter of the connection between the pores” refers to the diameter of the cross-section of the connection between two pores in the plane normal to the line connecting the centroids of the two pores, where the plane is chosen so that the area of the cross-section of the connection is at its minimum value. The term “diameter of a cross-section of a connection” refers to the average length of a straight line segment that passes through the center, or centroid (in the case of a connection having a cross-section that lacks a center), of the cross-section of a connection and terminates at the periphery of the cross-section. The term “substantially all the connections” refers to at least 90% of the connections in the biomaterial, such as at least 95% or at least 97% of the connections. The diameter and shape of the pores, as well as the connections between them, may be assessed using scanning electron microscopy, for example.

In certain embodiments, the peripheral skirt is more flexible than the transparent central core. By “more flexible,” it is intended that the Young's modulus, Shore D hardness, and/or tensile strength value of the peripheral skirt reflects a value indicative of greater flexibility relative to the transparent central core. In certain embodiments, the peripheral skirt is less transparent than the transparent central core. The peripheral skirt may be opaque. As used herein, “opaque” refers to a substantial portion of visible light reflected or absorbed by the skirt, such as greater than or equal to 80% of incident light. In certain embodiments, about or at least about 85%, 90%, 95%, or 99% or more of incident light is reflected or absorbed.

As noted above, a hydrogel refers to a three-dimensional polymer that measurably swells in an aqueous solution due to the absorbance of water. A polymer of the porous hydrogel may further comprise a functional group (e.g., carboxyl, amide, amino, ether, hydroxyl, cyano, nitrido, sulfanamido, acetylenyl, epoxide, silanic, anhydric, succinimic, azido, or any combination thereof). Such a functional group may permit attachment of other molecules to the polymer, as discussed in more detail below. In certain embodiments, the porous hydrogel comprises at least one terminal acetylenyl group. In certain embodiments, the porous hydrogel comprises at least one terminal azido group. A porous hydrogel may comprise, for example, multiple 1,2,3-triazolyl groups, each positioned between a first polymer and a second polymer, wherein the first and second polymers may be the same or different, and may each independently be a poly(ethylene glycol) (PEG). In certain embodiments, the first polymer comprises a group defined as —(OCH₂CH₂)ₘ—, wherein m=2-100. For example, m may be 2, 5, 10, 15, 20, 25, 30, 35, 40, 45, 50, 55, 60, 65, 70, 75, 80, 85, 90, 95, or 100, or at least or at most any of these values, or any range derivable therein. In certain embodiments, the second polymer comprises a group defined as —(OCH₂CH₂)ₙ—, wherein n=2, 5, 10, 15, 20, 25, 30, 35, 40, 45, 50, 55, 60, 65, 70, 75, 80, 85, 90, 95, or 100, or at least or at most any of these values, or any range derivable therein. A polymer may be PEG-diacrylate. A porous hydrogel may comprise silicone. In particular embodiments, a porous hydrogel comprises the following:

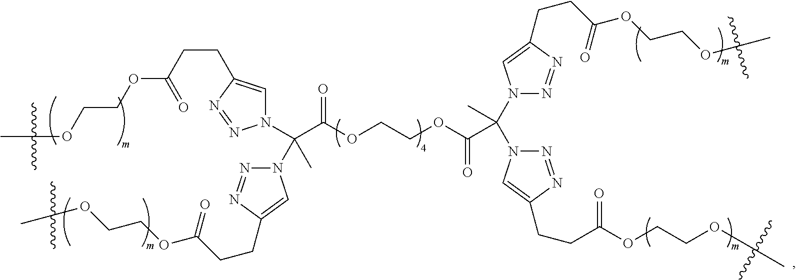

wherein m=2-100, as described above.

A peripheral skirt may further comprise a biomolecule or an antibiotic, or a combination thereof. As used herein, a “biomolecule” refers to any carbon containing molecule synthesized by a living organism. The biomolecule may comprise, for example, a nucleic acid, an amino acid, a peptide, a protein, a terpene, a fatty acid, a carbohydrate, a steroid, or an oil, or a combination thereof. In certain embodiments, the biomolecule is a protein, such as type I collagen, fibronectin, or TGF-β2. Any antibiotic known in the art may be employed, and non-limiting examples of antibiotics include gentamicin, vancomycin, and tobramycin.

The biomolecule or antibiotic may be attached or coupled to the porous hydrogel in any manner known to those of skill in the art, such as through a covalent bond. Coupling chemistries include, but are not limited to, the formation of esters, ethers, amides, azido and sulfanamido derivatives, cyanate, and other linkages to functional groups, such as those described above, available on the porous hydrogel. For example, the biomolecule or antibiotic may be covalently attached to the porous hydrogel through a 1,2,3-triazolyl group.

The biomolecule or antibiotic may also be introduced into the porous hydrogel by forming the porous hydrogel in the presence of the biomolecule or antibiotic, by allowing the biomolecule or antibiotic to diffuse into the porous hydrogel, or by otherwise introducing the biomolecule or antibiotic into the porous hydrogel.

The peripheral skirt may further comprise a polymer that was employed to generate the transparent central core. For example, the peripheral skirt may further comprise PMMA. In certain embodiments, the polymer partially or fully interpenetrates the peripheral skirt. By “partially interpenetrates,” it is meant that at least 1% of the polymer interpenetrates into the peripheral skirt at the boundary of the transparent central core and the peripheral skirt to form an interpenetrating polymer network (IPN).

The diameter of the peripheral skirt is, of course, larger than that of the transparent optical core. In certain embodiments, the peripheral skirt has a diameter ranging from between 9-13 mm. In certain embodiments, the diameter is about, at most about, or at least about 9, 10, 11, 12, or 13 mm, or any range derivable therein.

Methods of making a peripheral pre-form skirt of a keratoprosthesis are also provided by the present invention. Such a pre-form skirt may be used in a later step to produce a keratoprosthesis that further comprises a transparent central core. In certain embodiments, such a method comprises the step of forming a biocompatible hydrogel around a template comprising an array of monodisperse porogens. As used herein, the term “porogens” refers to any structures that can be used to create a template that is removable after the biocompatible polymer scaffold is formed under conditions that do not destroy the polymer scaffold. Exemplary porogens that are suitable for use in the methods of the invention include, but are not limited to, polymer particles such as PMMA beads and polystyrene beads. In certain embodiments, PMMA beads are employed.

The porogens may have any suitable shape that will permit the formation of a porous hydrogel with an array of pores, wherein substantially all the pores have a similar diameter, wherein the mean pore diameter is between 10-100 nm. For example, the porogens may be spherical. Other suitable porogen shapes include, but are not limited to, dodecahedrons (such as pentagonal dodecahedons) and ellipsoids. In some embodiments, substantially all the porogens have a roundness of at least about 0.1, such as at least about 0.3 or at least about 0.7. “Roundness” is defined as described above.

Substantially all the porogens in the array have a similar diameter. As used herein, the term “substantially all the porogens” refers to at least 90% of the porogens, such as at least 95% or at least 97% of the porogens. As used herein, “diameter of the porogen” is defined as the longest line segment that can be drawn that connects two points within the porogen, regardless of whether the line passes outside the boundary of the porogen.

Two porogens have a “similar diameter” when the difference in the diameters of the two porogens is less than about 20% of the larger diameter, such as about 15%, 10%, or 5%. A plurality of porogens having similar diameter may be termed “monodisperse porogens.” Porogens having similar diameters of a desired size may be obtained by size fractionation. Typically, the mean diameter of the porogens is between about 20 and about 90 nm, such as between about 25 and 75 nm or between about 30 and 60 nm. In some embodiments, the mean diameter of the porogens is between about 30 and 40 nm. In certain embodiments, the mean diameter of the porogens is about, at most about, or at least about 10, 15, 20, 25, 30, 35, 40, 45, 50, 55, 60, 65, 70, 75, 80, 85, 90, 95, or 100 nm, or any range derivable therein. Monodispersity may be assessed by light microscopy, for example.

In some embodiments, the invention provides methods for forming a template. As used herein, a “template” refers to a packing of a plurality of porogens. Formation of a template allows for later formation of a porous hydrogel, as will be explained. Methods of forming a template may comprise packing the porogens into a mold. Any mold may be used for packing the porogens. For example, a suitable mold may be formed using an upper mold and lower mold that are separated, such as shown in FIG. 3. The separation between the upper and lower mold may be, for example, 50, 55, 60, 65, 70, 76, 80, 85, 90, 95, 100, 105, 110, 115, 120, 125, 130, 135, 140, 145, 150, 155, 160, 165, 170, 175, 180, 185, 190, or 200 μm. The porogens may be packed into a mold using ultrasonic agitation, or any other suitable method for obtaining a closely packed array of porogens.

In some embodiments, methods for forming the template comprise fusing the porogens to form connections between the porogens. The porogens may be fused by sintering. Typically, the sintering temperature is higher than the glass transition temperature of the polymer, such as between about 10° C. and about 50° C. higher than the glass transition temperature of the polymer. As an example, PMMA porogens may be used, where PMMA has a glass transition temperature of about 105° C.: thus, the sintering temperature of PMMA typically ranges from about 115-165° C. Increasing the duration of the sintering step at a given temperature increases the connection size; increasing the sintering temperature increases the growth rate of the connections. Suitable sintering times are generally between 1 and 48 hours. Porogens can also be fused by other methods. For example, the porogens can be fused by partially dissolving them by treatment with a suitable solvent.

Once a template has been created, a hydrogel is formed around the template. The hydrogel may comprise any biocompatible polymer that permits formation of a hydrogel, such as synthetic polymers, naturally-occurring polymers, or mixtures thereof. In particular embodiments, the hydrogel comprises PEG, such as PEG having a molecular weight of about 2000-8000 Da. PEG-diacrylate may also be employed. The hydrogel may further comprise a biomolecule or an antibiotic, as described above.

In some embodiments, the hydrogel is formed around the template by polymerizing a polymer precursor mixture around the template. The polymer precursor mixture typically comprises polymer precursors and suitable cross-linking reagents, which are agents known in the art. See, e.g., U.S. Pat. No. 5,300,116, incorporated herein by reference in its entirety. This reference also describes suitable initiators as well. A polymer precursor mixture may comprise methyl methacrylate, for example.

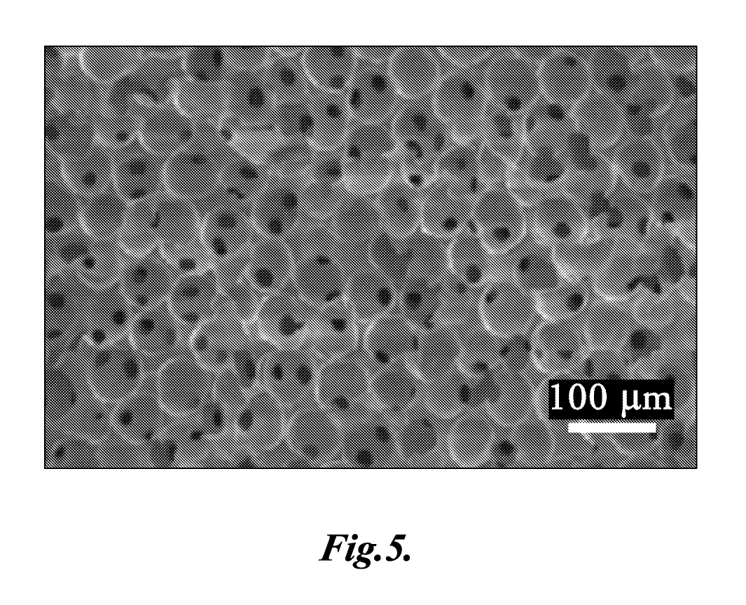

After a hydrogel has been formed, the template is removed to produce the porous hydrogel (see FIG. 5). In some embodiments, the template is removed by solvent extraction. For example, PMMA porogens may be extracted with a 9:1 v/v acetone-water solution.

In more specific embodiments, the present invention contemplates a method of making a peripheral pre-form skirt of a keratoprosthesis comprising a porous hydrogel, the method comprising: (a) heat sintering monodisperse PMMA porogens to create a template; (b) combining the template with a solution comprising monomers or a mixture of monomers; (c) polymerizing the solution; and (d) removing the template to reveal a porous hydrogel. A monomer may be ethylene glycol, for example. In certain embodiments, step (b) may entail combining the template with a solution comprising (i) a poly(ethylene oxide) diester comprising at least one terminal acetylenyl group with (ii) a poly(ethylene oxide) diester comprising at least one terminal azido group or a poly(ethylene oxide) diether comprising at least one terminal azido group. The poly(ethylene oxide) diester comprising at least one terminal acetylenyl group may be further defined as:

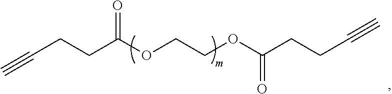

wherein m=2-100, as described above. The poly(ethylene oxide) diester comprising at least one terminal azido group may be further defined as:

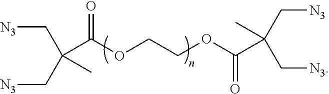

The poly(ethylene oxide) diether comprising at least one terminal azido group may be further defined as:

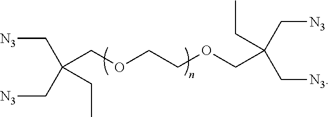

In any embodiment herein, the polymerization step may comprise reacting the solution with a copper(I)-containing catalyst. Methods of the present invention may further comprise removing substantially all of the copper(I) from the porous hydrogel, such as more than 90% of the copper(I). Removal may take place by sequential aqueous washings, for example.

In certain embodiments, a porous hydrogel of the present invention comprises the following polymer:

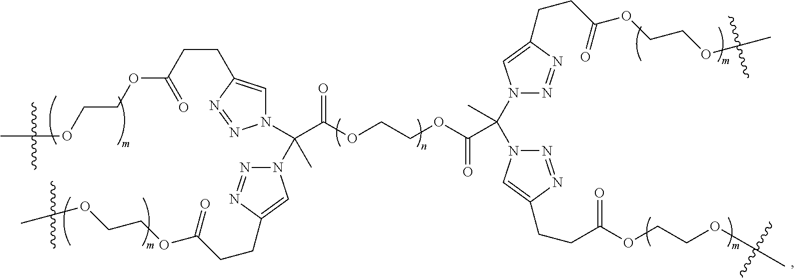

wherein m and n are each independently 2-100, as discussed above.

Any method discussed herein regarding formation of a porous hydrogel may further comprise attaching a biomolecule or an antibiotic to the porous hydrogel, as discussed above. Such an attachment may comprise, for example, a 1,2,3-triazolyl linkage.

In other embodiments, the present invention provides a keratoprosthesis consisting essentially of: (a) a rigid transparent central core; and (b) a peripheral skirt comprising a porous hydrogel. Non-limiting examples of aspects that would materially alter the properties of such a keratoprosthesis include imparting a rigidity to the peripheral skirt that would render it more rigid than the transparent central core; imparting a flexibility to the transparent central core that would render it less rigid than the peripheral skirt; causing the transparent central core to be a hydrogel; and introducing any physical separation between the transparent central core and the peripheral skirt.

In more particular embodiments, the present invention provides a keratoprosthesis consisting of (a) a rigid transparent central core; and (b) a peripheral skirt comprising a porous hydrogel.

In further embodiments, the present invention provides a keratoprosthesis consisting essentially of: (a) a rigid transparent central core consisting essentially of PMMA; and (b) a peripheral skirt comprising a porous hydrogel. Non-limiting examples of aspects that would materially alter the properties of such a keratoprosthesis include those aspects listed above.

In further embodiments, the present invention provides a keratoprosthesis consisting essentially of: (a) a rigid transparent central core consisting of PMMA; and (b) a peripheral skirt comprising a porous hydrogel.

Also contemplated by the present invention are methods of making a keratoprosthesis. Such methods may comprise, for example, (a) forming or placing a porous hydrogel in the periphery of an injection molding apparatus to form a peripheral pre-form skirt; (b) injecting a solution comprising a monomer or a mixture of monomers into the molding apparatus such that the solution occupies the area inside the skirt; and (c) polymerizing the monomer(s) to generate a transparent central core. A “suitable monomer” is one which, when polymerized, forms a material having one or more properties of the transparent central core as discussed above (e.g., transparency, a particular Young's modulus, Shore D hardness, and/or a tensile strength). The solution will typically comprise other agents to aid in polymerization, such as a crosslinking agent and an initiator. Alternatively, one may inject a polymer or mixture of polymers into the area inside the skirt, rather than performing polymerization after injection of one or more monomers, to generate the transparent central core. A polymer that yields a material having one or more properties of the transparent central core as discussed above may be used in such methods.

In more specific embodiments, methods of making a keratoprosthesis may comprise: (a) forming a porous hydrogel in the periphery of an injection molding apparatus to form a peripheral pre-form skirt; (b) injecting a composition comprising methyl methacrylate into the injection molding apparatus, wherein the composition occupies the area inside the skirt; and (c) polymerizing the methyl methacrylate to form a transparent central core. Alternatively, one may inject polymerized PMMA directly into the central core. In certain embodiments, the PMMA partially or fully interpenetrates into the peripheral pre-form skirt. Methods may also comprise removal of any residual monomers, initiators, or cross-linking agents, such as through washings and/or aqueous extractions. Sterilization of the keratoprosthesis may also take place using methods known to those in the art.

Methods of treatment are also contemplated by the present invention. In certain embodiments, there is provided a method of treating corneal blindness comprising implanting a keratoprosthesis as described herein into a patient. In such methods, the keratoprosthesis may be implanted in a process comprising suturing the peripheral skirt of the keratoprosthesis to the corneal tissue of the patient. Methods may further comprise trimming the peripheral skirt of the keratoprosthesis prior to implantation, such that an appropriate size is achieved for implantation.

Any method of treatment may further comprise a step of removing a diseased, damaged, or otherwise impaired cornea from a patient prior to implantation of the keratoprosthesis. Such methods are well-known to those of skill in the art. Methods of removal may comprise performance of a full thickness circular trephination in the center of the subject's cornea, followed by removal of the cornea button by, e.g., scissors or knife.

As used herein, the term a “patient” refers to a living mammalian organism, such as a human, monkey, cow, sheep, goat, dog, cat, rabbit, mouse, rat, guinea pig, or transgenic species thereof. In certain embodiments, the patient or subject is a primate.

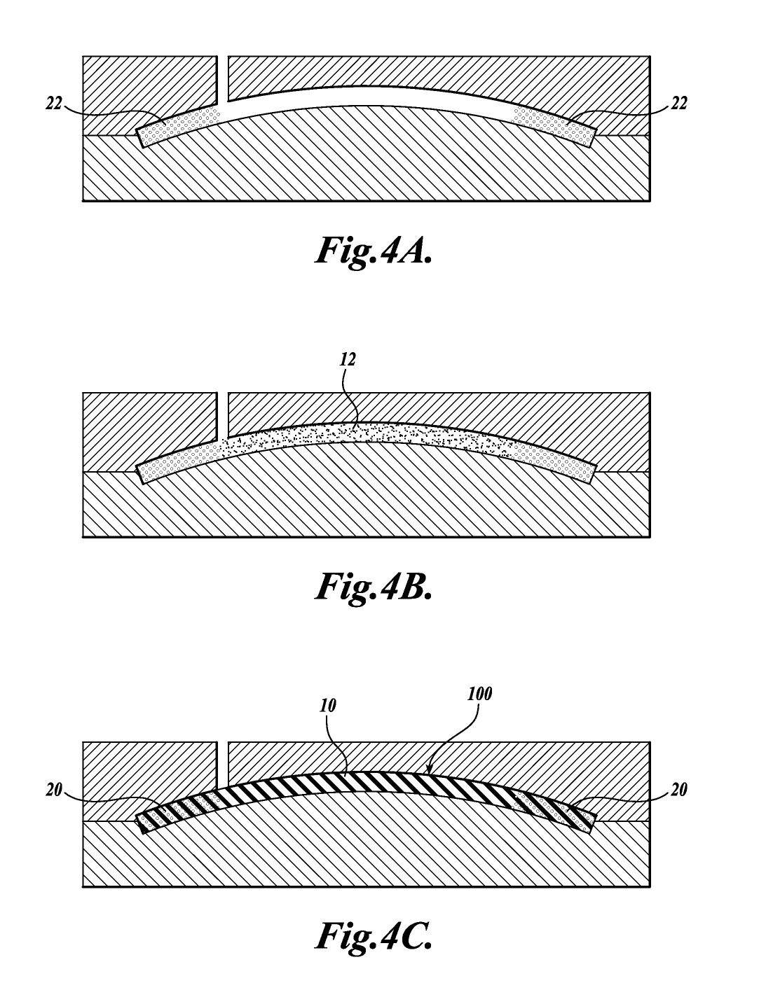

Certain embodiments of the present invention may be better understood by reference to the figures. FIG. 1A is a keratoprosthesis 100 of the present invention defined by a transparent central core 10 and a peripheral skirt comprising a porous hydrogel 20. FIG. 1A is a cross-sectional view of keratoprosthesis 100 showing the transparent central core 10 and the peripheral skirt comprising a porous hydrogel 20. Black hashmarks indicate that the material of transparent central core is polymerized. Areas of overlap of the black hashmarks over the peripheral skirt comprising a porous hydrogel 20 indicate an interpenetrating polymer network (IPN) generated during polymerization of the monomer material of the central core that interpenetrated into the peripheral skirt during polymerization (see FIGS. 4A, 4B and 4C). Here, polymerization has fully extended throughout the peripheral skirt.

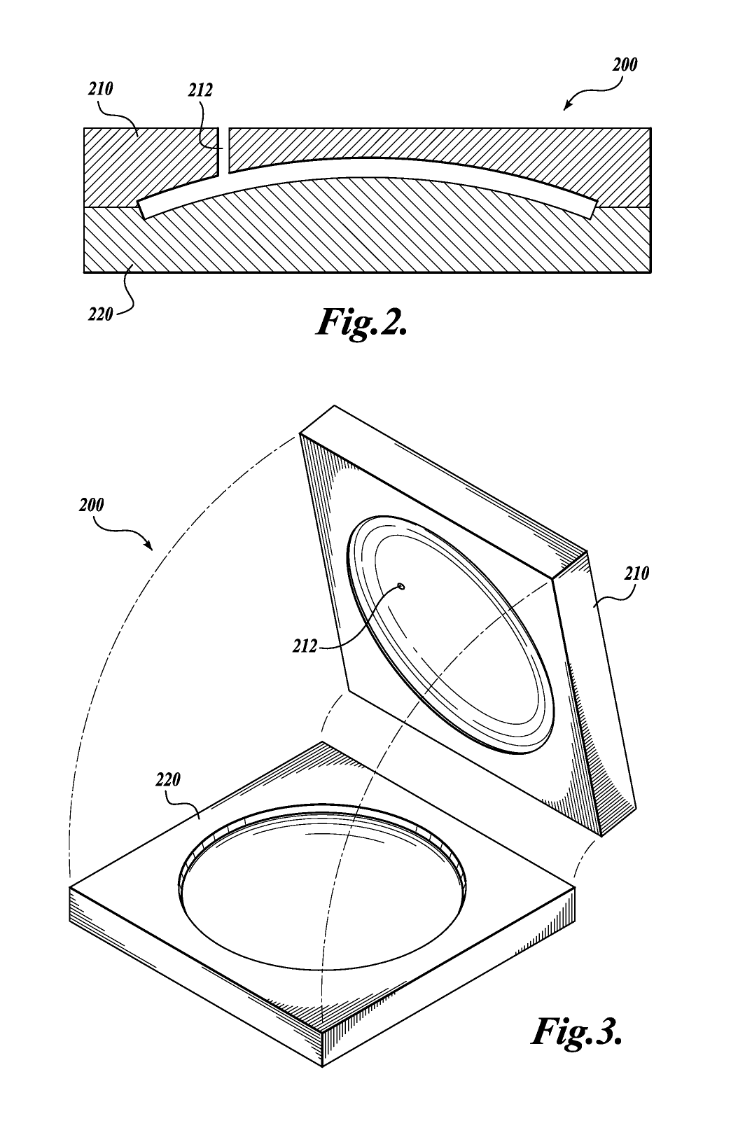

FIGS. 2 and 3 each show a molding apparatus 200 that may be used in the manufacture of a keratoprosthesis of the present invention. Each molding apparatus may be an injection molding apparatus. FIG. 2 is a cross-section of a molding apparatus 200 with an upper mold 210 and a lower mold 220. Opening 212 allows for introduction of materials, such as a porous hydrogel or a monomer that may be used to manufacture a transparent central core. FIG. 3 also shows an upper mold 210 and a lower mold 220 along with an opening 212.

FIGS. 4A, 4B and 4C are side views of a molding apparatus that may be used in the manufacture of a keratoprosthesis of the present invention. In FIG. 4A, a peripheral skirt comprising a porous hydrogel 22 is shown. In FIG. 4B, a monomer material 12 has been injected into the central core of the molding apparatus. Following polymerization of the monomer, the polymerized material 10 is shown in the finished keratoprosthesis 100 in FIG. 4C. As described in FIG. 1B, areas of overlap of the black hashmarks over the peripheral skirt comprising a porous hydrogel 20 indicate an IPN generated during polymerization of the monomer material of the central core that fully interpenetrated into the peripheral skirt during polymerization. The IPN may be full or partial, as discussed herein.

The use of the term “or” in the claims is used to mean “and/or” unless explicitly indicated to refer to alternatives only or the alternatives are mutually exclusive. It is specifically contemplated that any listing of items using the term “or” means that any of those listed items may also be specifically excluded from the related embodiment.

Throughout this application, the term “about” is used to indicate that a value includes the standard deviation of error for the device or method being employed to determine the value.

As used herein the specification, “a” or “an” may mean one or more, unless clearly indicated otherwise. As used herein in the claims, when used in conjunction with the word “comprising,” the words “a” or “an” may mean one or more than one.

The terms “comprise,” “have” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. For example, any method discussed herein may employ any keratoprosthesis described herein.

While illustrative embodiments have been illustrated and described, it will be appreciated that various changes can be made therein without departing from the spirit and scope of the invention.

The embodiments of the invention in which an exclusive property or privilege is claimed are defined as follows:

1. A keratoprosthesis, comprising:
(a) a rigid transparent central core; and
(b) a peripheral skirt comprising a porous hydrogel.

2. The keratoprosthesis of claim 1, wherein the rigid transparent central core has a Young's modulus ranging from about 500 to about 3500 MPa.

3. The keratoprosthesis of claim 1, wherein the rigid transparent central core has a Shore D hardness ranging from about 60 to about 100.

4. The keratoprosthesis of claim 1, wherein the rigid transparent central core has a tensile strength ranging from about 30 to about 70 MPa.

5. The keratoprosthesis of claim 1, wherein the rigid transparent central core comprises polymethylmethacrylate (PMMA).

6. The keratoprosthesis of claim 1, wherein the rigid transparent central core consists essentially of PMMA.

7. The keratoprosthesis of claim 1, wherein the rigid transparent central core has a diameter ranging from about 5 to about 8 mm.

8. The keratoprosthesis of claim 1, wherein the rigid transparent central core has a thickness ranging from about 50 to about 200 μm.

9. The keratoprosthesis of claim 1, wherein the rigid transparent central core has a diopter ranging from about 10 to about 70 diopters.

10. The keratoprosthesis of claim 1, wherein the peripheral skirt further comprises a biomolecule or an antibiotic.

11. The keratoprosthesis of claim 10, wherein the biomolecule is a protein.

12. The keratoprosthesis of claim 11, wherein the protein is type I collagen, fibronectin, or TGF-β2.

13. The keratoprosthesis of claim 10, wherein the biomolecule or antibiotic is covalently attached to the porous hydrogel.

14. The keratoprosthesis of claim 1, wherein the peripheral skirt further comprises PMMA.

15. The keratoprosthesis of claim 1, wherein the peripheral skirt has a diameter of about 9 to about 13 mm.

16. A method of making a peripheral pre-form skirt of keratoprosthesis comprising a porous hydrogel, the method comprising:
(a) heat sintering monodisperse poly(methyl methacrylate) porogens to create a template;
(b) combining the template with a solution comprising a monomer or mixture of monomers;
(c) polymerizing the solution; and
(d) removing the template to provide a porous hydrogel.

17. The method of claim 16, further comprising covalently attaching a biomolecule or an antibiotic to the porous hydrogel.

18. A method of making a keratoprosthesis, comprising:
(a) forming or placing a porous hydrogel in the periphery of an injection molding apparatus to form a peripheral pre-form skirt;
(b) injecting a solution comprising a monomer or a mixture of monomers into the injection molding apparatus, wherein the solution occupies the area inside the skirt; and
(c) polymerizing the monomer(s) to generate a rigid transparent central core.

19. The method of claim 18, wherein the monomer of step (b) is methyl methacrylate.

20. The method of claim 19, wherein PMMA formed from polymerizing the methyl methacrylate polymer penetrates into the peripheral pre-form skirt.

21. A method of treating corneal blindness, comprising implanting a keratoprosthesis of claim 1 into a patient.

22. The method of claim 21, wherein the keratoprosthesis is implanted in a process by suturing the peripheral skirt of the keratoprosthesis to the corneal tissue of the patient.
